(19) United States
(12) Patent Application Publication
(10) Pub. No.: US 2024/0200051 A1
(43) Pub. Date: Jun. 20, 2024

(54) CONTAMINANT DEGRADATION METHODS, BIOMATERIAL COMPOSITIONS, AND SYSTEMS

(71) Applicant: COLORADO SCHOOL OF MINES, Golden, CO (US)

(72) Inventors: Karla Diviesti, Golden, CO (US); Richard C. Holz, Golden, CO (US)

(21) Appl. No.: 18/544,148

(22) Filed: Dec. 18, 2023

Related U.S. Application Data
(60) Provisional application No. 63/387,877, filed on Dec. 16, 2022.

(51) Int. Cl. C12N 11/04; C02F 3/34; C02F 101/38; C02F 103/06; C12N 9/14; C12N 11/10

(52) U.S. Cl. CPC C12N 11/04; C02F 3/342; C12N 9/14; C12N 11/10; C12Y 308/01008; C02F 2101/38; C02F 2103/06

(57) ABSTRACT

Disclosed herein are compositions, methods, and systems useful in remediating contaminants, for example environmental contaminants. In many embodiments, the contaminant is selected from atrazine and chlorothalonil, and the remediation is accomplished with a biomaterial. In many embodiments, the biomaterial comprises an enzyme that catalyzes the contaminant remediation and a gel, for example a biogel or sol-gel. In some embodiments, the enzyme is triazine hydrolase (TrzN) for example TrzN from Arthrobacter aurescens TC1. The gel may be in the form of beads, for example alginate beads, and the beads may be coated, for example in chitosan. The disclosed sol-gel may be comprised of tetramethylorthosilicate (TMOS). The disclosed compositions, methods, and systems may increase enzymatic activity and/or protect the enzyme from activity degradation due to pH, temperature, proteolysis, dehydration, etc.

### CROSS-REFERENCE TO RELATED APPLICATIONS

This application claims benefit of priority pursuant to 35 U.S.C. § 119(e) of U.S. provisional patent application No. 63/387,877 entitled “Catalytic Biomaterials for Atrazine Degradation,” filed on 16 Dec. 2023, which is hereby incorporated by reference in its entirety.

### GOVERNMENT LICENSE RIGHTS

This invention was made with Government support under CHE-200241 awarded by the National Science Foundation. The Government has certain rights in the invention.

### FIELD

The disclosed processes, methods, and systems are directed to remediation of environmental contaminants, such as atrazine and chlorothalonil with stable and reusable biomaterials comprising encapsulated dehalogenases.

### BACKGROUND

Atrazine (2-chloro-4-ethylamino-6-isopropylamino-s-triazine) is a water-soluble herbicide that inhibits photosynthesis in targeted plants. Originally patented in 1958, commercial use in the U.S. began in 1959 and today it's the second most used herbicide, with roughly 30,000 tons applied annually to sorghum, sugarcane, and corn crops. Atrazine is persistent and mobile in aquatic environments, primarily through surface runoff where it enters groundwater via leaching. Once in the watershed, atrazine has the potential to travel miles as it has a half-life of six months to several years, which is troublesome considering the potential downstream effects. Atrazine toxicity impacts eukaryotes including, but not limited to, crustaceans, insects, mollusk, fish, amphibians, and reptiles. The effects observed in aquatic eukaryotes after atrazine exposure raises concerns for the possible effect's atrazine presents to humans, such as, lung and kidney diseases, cardiovascular damage, retinal degeneration, and cancer, which is why in 2003 the European union banned atrazine. In the US, the EPA Endocrine Disruptor Testing Advisory committee recognized atrazine as an endocrine toxin in humans in 2000 and shortly thereafter, the agency for Toxic substances and Disease Registry (ATSDR) warned that people in rural communities, near agricultural lands, are at an increased risk of atrazine exposure primarily through their drinking water. Given the widespread use of atrazine and its toxicity to both aquatic environments and humans, its biodegradation and environmental clean-up has become a topic of significant importance.

What is needed is a method of remediating TrzN that is cost-effective, efficient, and can be applied to remediation of other halogen-containing compounds.

### SUMMARY

Applicants describe herein, compositions, methods, and systems to overcome the challenges described above. In many embodiments, appropriate enzymes may be immobilized, for example encapsulated, in a biomaterial. In many embodiments, the disclosed biomaterial may have various beneficial properties, for example the biomaterial may be optically transparent, porous, may permit small molecules (for example contaminating compounds and substrates) access to the immobilized enzyme, while maintaining the solution structure and native function of the enzyme.

Disclosed herein is the immobilization of purified TrzN within an alginate matrix in the absence and presence of a chitosan outer layer, as well as immobilization of TrzN within silica glasses derived through the sol-gel process. These novel biocatalytic materials are capable of supporting hydrolytic dechlorination of atrazine into its less toxic derivative hydroxyatrazine under mild conditions.

### BRIEF DESCRIPTION OF THE DRAWINGS

FIG. 1 depicts the use of biocatalyst triazine hydrolase (TrzN) in dechlorination of atrazine to hydroxyatrazine.

FIG. 2 is a graph of atrazine concentration as a function of time for various embodiments such as TrzN:sol-gel, TrzN:alginate, and TrzN:chitosan.

FIG. 3 are four scanning electron micrograph (SEM) images of various embodiments. Panel A is an SEM image of TrzN:alginate (left) biomaterial at 200× magnification. The scale bar shows 300 μm. Panel B is an SEM image of TrzN:alginate (left) and TrzN:chitosan (right) biomaterial at 235× magnification. The scale bar shows 300 μm. Panel C is an SEM image of TrzN:sol-gel biomaterial at 510× magnification. The scale bar shows 100 μm. Panel D is an SEM image of cross-sectional view of dried TrzN:alginate biomaterial at 660× magnification. The scale bar shows 100 μm.

FIG. 4 is a bar graph showing % activity at 0.5 and 18 hours for various embodiments including TrzN, TrzN:sol-gel, TrzN:alginate, and TrzN:chitosan biomaterials proteolytically digested with trypsin. The soluble TrzN and the biomaterials were incubated at a 2:1 ratio of trypsin:TrzN at 35° C. TrzN:alginate, and TrzN:chitosan were digested for 30 minutes while TrzN:sol-gel was digested for 18 hours. A soluble TrzN control was digested for both a 0.5 and 18 hour period. A “*” symbol indicates there was no observable activity.

FIG. 5 is a bar graph showing % activity versus temperature for various embodiments including TrzN, TrzN:alginate, and TrzN:chitosan biomaterials. The residual activity of the soluble enzyme and biomaterials was recorded after a 30 minute heat shock in a 50°, 60°, 70°, and 80° C. water bath followed by measuring the dechlorination of atrazine at 25° C. for 6 hours. A “*” indicates there was no observable activity.

FIG. 6 is a graph showing % activity versus time for various embodiments including TrzN, TrzN:alginate, TrzN:chitosan, and TrzN:sol-gel. Values were calculated with the assumption of complete protein retention over the 6 week experiment. The kcat of soluble TrzN was evaluated with 150 μM atrazine at 25° C. then stored in 0.1 M sodium phosphate buffer pH 7.0 at 4° C. until the following week. The TrzN:alginate, TrzN:chitosan, and TrzN:sol-gel biomaterials were evaluated weekly for the degradation of atrazine in 50 mM atrazine in 50 mM HEPES pH 7.0 for their respective running conditions. The biomaterials were washed and stored at 4° C. in 50 mM HEPES pH 7.0 for 1 week before repeating the experiment.

FIG. 7 is a bar graph showing % activity versus methanol concentration for various embodiments including soluble TrzN, TrzN:alginate, TrzN:chitosan, and TrzN:sol-gel for the conversion of atrazine to hydroxyatrazine in 5:95, 10:90, and 20:80 MeOH:water (v/v) co-solvent solutions.

FIG. 8 is a bar graph showing % activity versus pH for various embodiments including soluble TrzN, TrzN:alginate, and TrzN:chitosan for the conversion of atrazine to hydroxyatrazine in 50 mM citric acid pH 4.0 buffer and 50 mM glycine buffer pH 9.0. A “*” indicates there was no observable activity.

### DETAILED DESCRIPTION

Disclosed herein are methods and systems for remediation and degradation of atrazine and atrazine-contaminated compositions.

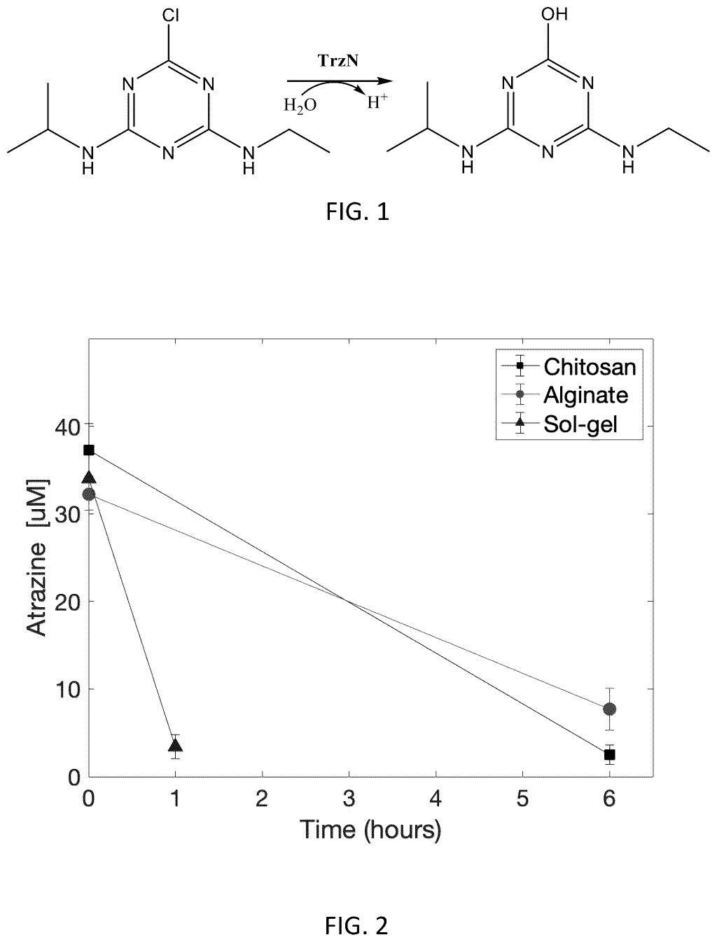

Triazine hydrolase (TrzN, EC 3.8.1.8) from Arthrobacter aurescens TC1 has great potential as a biocatalyst for the bioremediation of atrazine because it irreversibly catalyzes the dechlorination of atrazine to its less toxic derivative hydroxyatrazine under physiological conditions (FIG. 1). However, when utilizing enzymes for remediation purposes a major concern arises in one's ability to separate the enzyme from the reaction mixture. A related issue involves a protic solvent contaminants typically used for extraction, which degrades bacterial cells and significantly decreases the activity of most free enzymes, including TrzN. Bioremediation methods utilizing TrzN have been limited to whole cells that naturally express TrzN or in one case by simply adding TrzN to a contaminated drainage ditch. While these were successful in degrading atrazine, recovering whole cells or free enzymes from the environment pose major obstacles as well as thermal and proteolytic degradation. Side reactions within a cell also pose problems.

Applicant shows successful encapsulation of triazine hydrolase from Arthrobacter aurescens TC1 (TrzN) in alginate beads (TrzN:alginate), alginate beads coated in chitosan (TrzN:chitosan), and tetramethylorthosilicate (TMOS) gels using the sol-gel method (TrzN:sol-gel). TrzN:alginate and TrzN:chitosan hydrolyzed 50 μM Atrazine in 6 hours with negligible protein loss and ˜80% conversion rate. TrzN:sol-gel exhibited a 6-fold increase in activity requiring only an hour digestion time to achieve >95% conversion with negligible protein loss. Treatment of the biomaterials with trypsin confirmed the catalytic activity is due to the encapsulated enzyme and not surface bound TrzN. All the biomaterials showed potential for long-term storage with the only limitation arising from the loss of protein in storage buffer not denaturation of the encapsulated TrzN. TrzN:sol-gel stood out with ˜100% activity being retained after 10 consecutive reactions. Additionally, the materials stayed active in methanol concentrations <10% suggesting the potential to supplement reaction mixtures with organic co-solvents to increase the solubility of atrazine. The structurally integrity of the TrzN:alginate and TrzN:chitosan materials became limiting in extreme pH conditions while TrzN:sol-gel out performed soluble TrzN in these conditions. These data are the first example of TrzN being encapsulate within a biomaterial. These biomaterials are building blocks for the development of atrazine remediation strategies incorporating active TrzN within catalytically favorable materials.

### INTRODUCTION

Chlorinated aromatic hydrocarbons such as polychlorinated biphenyl and chlorobenzenes are important industrial starting materials for the manufacture of dyes and drugs, for example, while chlorothalonil and atrazine are a fungicide and pesticide, respectively. Aromatic carbon-chlorine bonds are typically very stable and hence, these compounds persist in soil and contaminant groundwater. Their low water solubility coupled with their toxicity, make them particularly important targets for environmental remediation. Enzymatic dehalogenation of chlorinated natural organic matter, compounds linked to cycling and possible respiration in organohalide-respiring bacteria such as Pseudomonas sp. CTN-3, in general, provides more soluble forms that are less likely to bioaccumulate and more likely to be susceptible to further degradation or cellular recycling and thus, less toxic. Therefore, cleavage of aromatic carbon-chlorine bonds is useful for the degradation of toxic industrial compounds.

Presented herein are methods to immobilize dehalogenases in, within, or with a biomaterial. In some embodiments, the dehalogenases triazine hydrolase (TrzN) and chlorothalonil dehalogenase (Chd) may be used. Both of these specific dehalogenases catalyze the irreversible dehalogenation of chlorinated aromatic hydrocarbons. The disclosed immobilization compositions, methods, and systems described herein are applicable to any dehalogenase enzyme. TrzN and Chd are exemplary systems that can be immobilized within functionalized biomaterials and can maintain their catalytic activity including substrate recognition, similar to the soluble enzyme. There are currently no methods of chlorothalonil or atrazine removal that utilizes an immobilized enzyme. The presently disclosed immobilization allows for the recovery of the enzymes and protection of them from proteolytic digestion. Bioremediation methods, using the disclosed biomaterials are also disclosed.

The presently disclosed biomaterial-immobilized dehalogenases provide for novel methods of encapsulating enzymes, useful in removing contaminants, for example atrazine or chlorothalonil, from various environments. In many of these environments, a dehalogenase may be useful for removing these contaminants, for example aquatic environments. The presently disclosed compositions, methods, and systems may be useful in various different bioremediation applications and strategies. Described herein is the successful immobilization of TrzN and Chd in alginate, chitosan coated alginate, sol-gels, and mesoporous silica nanoparticles (MSN). All of these biomaterials contain active enzymes that degrade halogens, for two examples atrazine or chlorothalonil. The present biomaterials may be useful in various bioremediation devices and systems, for example in continuous-flow packed bed bioreactors. In other embodiments, the biomaterials may be used in a batch bioreactor. The disclosed biomaterial compositions are dynamic and applicable to multiple bioremediation applications.

Current remediation strategies for atrazine using TrzN rely on whole microbial cells that naturally express TrzN or free TrzN. These methods are problematic in that there is no process to recover the cells, enzymes, and/or catalysts. Moreover, these materials are susceptible to environmental conditions which may degrade the enzyme, cell, or catalyst. Other methods for contaminant removal, for example atrazine removal, that do not use an enzyme catalyst include, phytoremediation, vermiremediation, photolysis, fenton chemistry, photocatalysis, plasma oxidation, electrokinetic process, reversible electrokinetic adsorption barrier (REKAB), nano zero-valent ion, activated carbon, and biochar adsorption. However, these methods may be costly, consume large amounts of energy, require toxic chemicals, and/or are not reusable. No biomethods exist for chlorothalonil degradation.

This work presents studies of the first immobilization of TrzN in any biomaterial. Alginate was chosen as the component of the first biomaterial due to its well documented properties and ease of construction. Chitosan coated alginate was also tested as a potential solution to the material limitations that can accompany alginate matrices. Lastly, TMOS sol-gel was evaluated as another potential biomaterial candidate due to being unique from the alginate matrices and a known material for a wide range of applications. The resulting biomaterials (TrzN:alginate, TrzN:chitosan, and TrzN:sol-gel) exhibited catalytic activity toward atrazine. All the biomaterials protected the soluble enzyme from proteolytic digestion. When evaluating reusability, TrzN:alginate remained active for 4 weeks and TrzN:chitosan remained active for 6 weeks. The chitosan coating prevented swelling resulting in less leakage which enabled the TrzN:chitosan biomaterial to remain active longer than the TrzN:alginate biomaterial. TrzN:sol-gel remained active for the full 6 weeks and showed higher activity retention than TrzN:chitosan. The soluble enzyme remained active for the full 6 weeks as well. The activity loss in the biomaterials can be explained by the loss of enzyme within the matrices not the denaturation of the enzyme. TrzN:sol-gel was also evaluated in a cyclical reuse study and showed no activity loss over 10 continuous cycles. This accentuated TrzN:sol-gel's potential to be reused multiple times making it a good candidate for bioremediation strategies. In a methanol co-solvent, soluble TrzN remains nearly fully active up to ˜99% methanol. Through 10% methanol, TrzN:alginate and TrzN:chitosan remained >75% active while TrzN:sol-gel retained ˜65% activity. This suggests the potential to supplement a reaction solution with methanol to increase atrazine solubility allowing higher concentrations of atrazine to be hydrolyzed. For TrzN:alginate and TrzN:chitosan, the most material limitations were related to pH dependence and thermostability. Both TrzN:alginate and TrzN:chitosan are pH dependent polymers and require an operating pH near neutral to prevent tightening of the polymer chain resulting in inactivity. In the same pH conditions TrzN:sol-gel showed no material limitations and outperformed both other biomaterials as well as the soluble TrzN. The thermostability analysis highlighted soluble TrzN's potential of hyper activity at higher temperatures. The TrzN:alginate material lost all activity at temperatures >60° C. while the TrzN:chitosan material was active through all 30 minute heat shocks, it was still significantly less active than the soluble enzyme. TrzN:sol-gel was able to withstand heat shocks >30 minutes at 50° C. and 60° C. All activity was lost at 70° C. Soluble TrzN remained active through 70° C. TrzN:sol-gel showed superior thermostability compared to alginate based materials but did not show improvement compared to the soluble enzyme. The soluble enzyme's extreme thermostability requires further experiments to understand its full potential. Overall, as a biocatalyst, TrzN:sol-gel proved to be the most effective. The TrzN:chitosan biomaterial provided more stability and longevity than the TrzN:alginate biomaterial. However, both materials showed degradation and structural shifts which prevented the immobilized TrzN from outperforming or performing as well as the soluble TrzN. The alginate and alginate coated chitosan biomaterials are simple and effective materials at providing the foundation for TrzN's ability to be catalytically active in an immobilization material. Moreover, the TrzN:sol-gel biomaterial was an effective biocatalyst—converting the chlorinated atrazine into its less toxic derivative hydroxyatrazine as well as or better than soluble TrzN. Under the right conditions, all these versatile functional biomaterials can be capable of degrading atrazine thus providing a new potential bioremediation strategy.

The disclosed enzymes may be immobilized by or to various materials. In most embodiments, the disclosed material may aid in protecting the stability, activity, structure, etc. of the protein. In some embodiments, the materials may be selected from one or more of alginate, chitosan, tetramethylorthosilicate (TMOS), etc. In some embodiments, the material may be in the form of beads, in one example alginate beads, and the beads may be coated with one or more compounds, for example chitosan, which may be useful in protecting the material and the immobilized enzyme, while allowing the contaminant access to the enzyme. Where triazine is immobilized with or within alginate, the composition may be referred to as TrzN:alginate. In some embodiments, TrzN:alginate may be coated with chitosan to form TrzN:chitosan. In embodiments where TMOS is a material, the TMOS may be formed into a gel, for example, in one embodiment by using the sol-gel method. This may result, after immobilization of triazine, in a material referred to as TrzN:sol-gel.

The disclosed material may support interaction between the immobilized enzyme and its substrate. In most embodiments the disclosed immobilized enzyme may support at least 80% conversion of its target substrate in a given time, for example from about 0.5 hr to 18 hr for example about 6-8 hrs. In many embodiments, the conversion of substrate may be more than 80%, for example 80%, 85%, 90%, 91%, 92%, 93%, 94%, 95%, 96%, 97%, 98%, of 99%, and less than about 100%, 99%, 98%, 97%, 96%, 95%, 90%, 85%, 80%, 75%, or 70%. In some embodiments, without wishing to be limited by theory, degradation of substrate material may be governed by mass transport. In some embodiments, mass transport may be affected by the porous materials and travel of the substrate through the porous materials to the enzyme active site.

### Contaminants

Contaminants, as used herein, may describe molecules and compounds, especially synthetic compounds used in agriculture that may have detrimental effects on human subjects. In many embodiments, the contaminants may be chlorinated compounds, for example chlorinated aromatic hydrocarbons such as polychlorinated biphenyl and chlorobenzenes. In many embodiments, the contaminants may be starting materials for the manufacture of dyes, drugs, fungicides, pesticides, etc. The contaminants may include aromatic carbon-halogen bonds that are highly stable. The disclosed contaminants may persist in soil and enter groundwater.

Contaminants may be treated to remove them and/or lower their concentration in the environment. In some embodiments, the contaminants may be subjected to enzymatic dehalogenation and rendered less toxic. In many embodiments, the contaminants are chlorothalonil and/or atrazine—a fungicide and a pesticide, respectively.

### Triazine Hydrolase

As used herein, triazine hydrolase may be used to refer to an enzyme with the ability to hydrolyze atrazine to produce hydroxyatrazine and chloride. Triazine hydrolase may also be referred to by other names, such as atrazine chlorohydrolase, atrazine dechloroinase. Triazine hydrolase may be isolated from various organisms, including, without limitation, bacteria, archaea, and eukaryota. In one example the enzyme is isolated from Arthrobacter, for example Arthrobacter aurescens or pseudomonas.

### Chlorothalonil Dehalogenase

Chlorothalonil dehalogenase, Chd, CTN-3 (EC 3.8.1.2) is a Zn(II)-dependent hydrolytic dehalogenase. Chd substitutes an aromatic chlorine on chlorothalonil with an aromatic alcohol to yield 4-hydroxytrichloro-isophthalonitrile, 4-dOH-TPN.

### Encapsulation

As used herein, encapsulation, may be used to refer to various techniques that protect and/or stabilize an enzyme without negating the enzyme's activity. In many embodiments, the encapsulating material may be a gel, in some embodiments a biogel or a synthetic gel, for one example alginate. In some embodiments, the gel may be further encapsulated within a coating, for example a bio or synthetic coating, in one example, chitosan. In various embodiments, the enzyme may be encapsulated within a sol-gel, for one example a tetramethylorthosilicate sol-gel. Other encapsulation materials include encapsulation within mesoporous silica nanoparticles (MSN).

Encapsulation may result in protection of the enzyme from various environments. In one example, encapsulation may result in the enzyme being inaccessible to large molecules for example a protease, it does not exclude the possibility that some enzymes may be at least partially accessible. In most embodiments, the majority of encapsulated enzymes may be inaccessible—for example greater than about 75%, 80%, 90%, 95%, 96%, 97%, 98%, or 99% of the encapsulated enzyme is inaccessible to protease.

Encapsulation and or immobilization by or within the material may aid in retaining enzyme activity and/or increasing activity relative to the same amount of soluble enzyme. Where less than 100% of the enzyme activity is retained, the amount may be less than about 99%, 98%, 97%, 96%, 95%, 94%, 93%, 92%, 91%, 90%, 85%, 80%, 75%, 70%, 65%, 60%, or 55%, and more than about 50%, 55%, 60%, 65%, 70%, 75%, 80%, 85%, 90%, 95%, 96%, 97%, 98%, or 99%. In some embodiments, enzyme activity may, with encapsulation, increase between about 1%, 2%, 3%, 4%, 5%, 6%, 7%, 8%, 9%, 10%, 20%, 30%, 40%, 50%, 60%, 70%, 80%, 90%, 100%, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, or 9.0-fold, and less than about 10-fold, 9-fold, 8-fold, 7-fold, 6-fold, 5-fold, 4-fold, 3-fold, 2-fold, 90%, 80%, 70%, 60%, 50%, 40%, 30%, 20%, or 10%. Retained activity may depend, in some cases, on the duration of digestion—in some embodiments, the duration may be up to about 18 hours. Changes in enzyme activity may be due to changes in catalytic activity (such as co-factor effects or mass transport) and/or changes in enzyme concentration (such as enzyme degradation or loss of enzymes).

Biomaterials may be effective at protecting the disclosed enzymes. In many embodiments, the disclosed biomaterials may sequester, hide, encapsulate, or shelter the disclosed enzymes such that the enzymes are not accessible to large molecules, for example soluble proteases. In some embodiments, treatment of the immobilized enzymes with soluble protease does not significantly reduce the biomaterial's enzymatic activity. In one embodiment, subjecting the immobilized enzymes to soluble trypsin may reduce enzyme activity to about 75% or more of their original activity, whereas trypsin digestion of free enzyme may reduce its activity to about 55% of its original activity. In most embodiments, the majority of encapsulated enzymes' activity may be inaccessible to protease—for example greater than about 75%, 80%, 90%, 95%, 96%, 97%, 98%, or 99% of the encapsulated enzyme may be inaccessible to protease.

The disclosed immobilized enzymes may be more thermostable than free enzymes. In some embodiments, immobilization of the disclosed enzymes to a support material (e.g. alginate, TMOS, etc.) may preserve stability over 25° C., for example over a temperature range of 30° C.-80° C. In many embodiments, immobilization of the disclosed enzymes may result in the enzyme retaining about 30% or more of its original activity at a temperature over about 30° C. for about a 30 min incubation period or longer. In some embodiments, the disclosed enzymes may retain greater than about 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99%, and less than about 100%, 99%, 98%, 97%, 96%, 95%, 94%, 93%, 92%, 91%, 90%, 85%, 80%, 75%, 70%, 65%, 60%, 55%, 50%, 45%, 40%, or 35% activity, at a temperature greater than about 90° C., 85° C., 80° C., 75° C., 70° C., 65° C., 60° C., 55° C., 50° C., 45° C., 40° C., 35° C., 30° C., or 25° C., and greater than about 20° ° C., 25° C., 30° C., 35° C., 40° C., 45° C., 50° ° C., 55° C., 60° C., 65° C., 70° C., 75° C., 80° C., or 85° C., and over a time period greater that about 1 min, 2 min, 3 min, 4 min, 5 min, 6 min, 7 min, 8 min, 9 min, 10 min, 15 min, 20 min, 25 min, 30 min, 40 min, 50 min, 60 min, 90 min, 2 hr, 3 hr, and less than about 2 hr, 90 min, 60 min, 55 min, 50 min, 45 min, 40 min, 35 min, 30 min, 25 min, 20 min, 15 min, 10 min, 9 min, 8 min, 7 min, 6 min, 5 min, 4 min, 3 min, or 2 min.

The disclosed biomaterial comprising immobilized enzyme is reusable and displays long-term stability compared to soluble enzymes. In many embodiments, the disclosed enzymes may retain activity over an extended duration. In many embodiments the amount of retained activity may be 65% or more over a duration greater than about 1 week, 2 weeks, 3 weeks, 4 weeks, 5 weeks, 6 weeks, 7 weeks, or 8 weeks, and less than about 10 weeks, 9 weeks, 8 weeks, 7 weeks, 6 weeks, 5 weeks, 4 weeks, 3 weeks, or 2 weeks, for one example greater than 5 weeks or 6 weeks. Without wishing to be limited by theory, loss in activity may be due, at least in part, to loss of enzyme from the biomaterial, rather than denaturation of the enzyme or product build up. In many embodiments, the immobilized enzyme may be reused multiple times, for example more than 2, 3, 4, 5, 10, 15, 20 times and less less than 50, 40, 30, 20, 15, 10, 5, 4, 3, or 2 times. In many embodiments, the disclosed enzymes may experience little or no loss of activity over ten reactions or more—for example the retained activity over that 10 weeks may be greater than 80% of the original activity.

The disclosed enzymes of the biomaterial may retain stability in various solutions and solvents. In one embodiment, the disclosed enzymes may retain stability in organic solvents, for example a protic solvent such as alcohol, at high concentrations. In one embodiment, the solvent is methanol (MeOH), and the methanol may be mixed with water at a 1:20 to 1:1 ratio of MeOH:water, for example from about 5:95 to about 20:80 MeOH:water mixtures, for example about 10:90. At various methanol concentrations, the immobilized enzymes may retain 5% or more of the activity of soluble enzyme, for example greater than about 10%, 20%, 70% or more. In some embodiments, the immobilized enzyme may retain about 5% or greater of its activity in an organic solvent solution, for example a 70:30 MeOH:water solution. Without wishing to be limited by example, in some cases, decreases in activity due to the presence of organic solvents may correlate with increased cation concentration in the reaction solution, for example Ca2+, or a reduced hydrodynamic volume of the porous biomaterial, which may restrict access to the enzyme.

The disclosed enzymes of the biomaterial may display enhanced stability over a range of solution conditions. In one embodiment, the disclosed enzymes may possess enhanced stability or activity at non-physiological pH. In many embodiments, the biomaterial-immobilized enzymes may retain activity at pH from about 4 to about 9. In many embodiments, the enzymes may exhibit at least 20% residual activity at non-physiologic pH, for example less than about 99%, 98%, 97%, 96%, 95%, 94%, 93%, 92%, 91%, 90%, 85%, 80%, 75%, 70%, 65%, 60%, or 55%, and more than about 50%, 55%, 60%, 65%, 70%, 75%, 80%, 85%, 90%, 95%, 96%, 97%, 98%, or 99%. In some embodiments, greater than about 20%, 25%, 30%, 40%, 45%, or 95% of original activity. Without wishing to be limited by theory, the disclosed biomaterial-immobilization may provide the enzyme with significant stabilization against both increased and decreased proton ion concentrations, which may result from electrostatic interaction between the biomaterial and enzyme.

### Biomaterial

As used herein, biomaterial may refer to the combination of material and enzyme, for example a protein or peptide encapsulated in at least one gel. In many embodiments, the protein or peptide is an enzyme, for example a hydrolase or dehalogenase. In some embodiments, the enzyme is a bacterial enzyme. In one embodiment, the enzyme is selected from triazine hydrolase and chlorothalonil dehydrogenase. In many embodiments, the atrazine hydrolase may be encapsulated in alginate and the alginate coated with a coating material, for example chitosan. The gel may be selected from various materials including sol-gels. In one embodiment the gel is alginate or tetramethylorthosilicate.

The disclosed biomaterials may be produced in various ways. In some embodiments, the disclosed biomaterials may be comprised of gel material, for one example alginate. In other embodiments, the biomaterial may be produced by a sol gel process. In some embodiments, the biomaterial may include a coating, for one example chitosan. Alginate biomaterials may be made in various ways, known to those of skill in the art. In one example, triazine may be mixed with a solution of sodium alginate powder for example a solution of about a 1% (w/v) alginate to buffer. In embodiments where the TrzN:alginate beads are coated, the coating may be chitosan. In these embodiments, a chitosan solution may be prepared by dissolving chitosan in water. Glacial acetic acid may be added to facilitate the dissolution. The chitosan solution may be filtered, and the concentration adjusted to about 0.8% (w/v). TrzN:Alginate beads may be added to the solution to facilitate coating.

Embodiments wherein the material is a sol-gel, the sol may include TMOS. In these embodiments TMOS, nanopure H₂O, and 0.040 M HCl may be combined and sonicated on ice. TMOS sol may be mixed with an equal volume of TrzN and then iced until gelation creates TrzN:sol-gel biomaterial in the form of monoliths. Monoliths can be washed, stored, and or crushed to create a heterogenous biomaterial.

Various enzymes may be immobilized with the disclosed materials to form the disclosed biomaterial. In many embodiments, the disclosed enzymes may be soluble enzymes, for example, soluble enzymes derived from bacterial sources. In various embodiments, the enzyme may dehalogenate a chlorinated aromatic compound, for example a dehalogenase. In some embodiments, the enzyme may be selected from triazine (TrzN) and chlorothalonil dehalogenase (Chd). In some embodiments, the enzyme may be an engineered enzyme. Engineered enzymes may have amino acid sequences that are less than 100% and more than about 80% identical to a wild-type or un-engineered (unmodified) enzyme. In some embodiments, the disclosed enzymes may be engineered to have one or more beneficial traits or one or more moieties to aid purification and/or interaction with the material of the biomaterial. In some embodiments, the enzyme may be engineered at the C- or N-terminal terminus. In one embodiment, the moiety is a hexa-histidine (His6 or 6-His) affinity tag, which may aid in purifying the enzyme via metal affinity chromatography (IMAC).

### EXAMPLES

### Example 1—Encapsulation of TrzN

Soluble TrzN was obtained by overexpression of the gene from Arthrobacter aurescens TC1 with an engineered N-terminal a hexa-histidine (His6) affinity tag in E. coli that was synthesized with optimized E. coli codon usage. Per liter of culture, ˜9 mg of pure TrzN is obtained after a single purification step utilizing immobilized metal affinity chromatography (IMAC). SDS gel page reveals a single polypeptide band at ˜51 kDa, consistent with previous studies and size exclusion chromatography indicates that TrzN exists primarily as a dimer (˜102 kDa) in solution in 50 mM HEPES buffer, pH 7.0, at 25° C. Purified TrzN was found to catalyze the hydrolysis of atrazine with a kcat value of 4.0±0.1 s−1 and a Km value of 43±3 μM in 50 mM HEPES buffer at pH 7.0 and 25° C. using a continuous spectrophotometric assay that monitors atrazine degradation at 264 nm (ε264=3.5 mM−1 cm−1). These values are consistent with those previously reported.

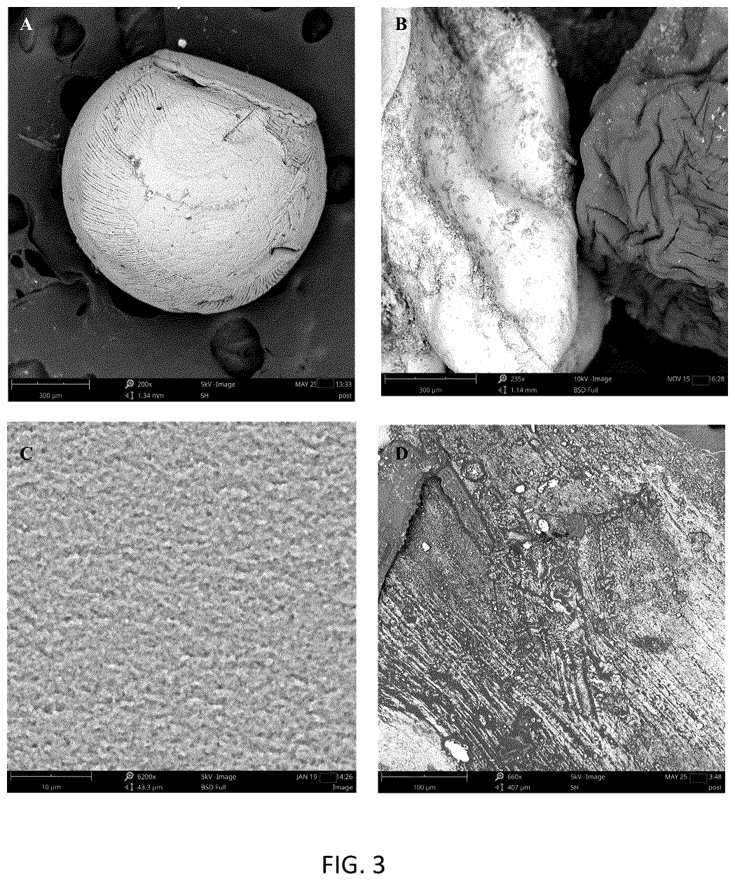

Having pure, catalytically active TrzN in hand allowed for its encapsulation into alginate beads (TrzN:alginate), TrzN:alginate coated with chitosan (TrzN:chitosan), and tetramethylorthosilicate (TMOS) gels using the sol-gel method (TrzN:sol-gel). Analysis of both the buffer in which the biomaterials were stored and the buffer from all the washing steps using the Bradford assay indicated that on average <0.5% of the TrzN used was present in either the wash buffer or the storage buffer. These data indicate that >99% of the TrzN present is encapsulated within the materials. Interestingly, the TrzN:alginate, TrzN:chitosan, and TrzN:sol-gel biomaterials readily react with atrazine (FIG. 2) at 25° C. in 50 mM HEPES buffer, pH 7.0 with ˜80% conversion of atrazine to the non-toxic hydroxyatrazine product over a 6-hr. reaction for TrzN:alginate and TrzN:chitosan while 100% degradation of atrazine by TrzN:sol-gel was observed after only 1-hr, a six-fold improvement for the degradation of atrazine comparted to the alginate biomaterials. These data indicate that the kinetics of substrate turnover for the TrzN:alginate, TrzN:chitosan, and TrzN:sol-gel biomaterials appear to be governed by mass transport of the substrate through the porous materials to the enzyme active site. SEMs of each of these biomaterials show the size of the beads and the porous nature of the surface confirming solution and substrate access to the encapsulated enzyme (FIG. 3). In FIG. 3B-C, the SEM of TrzN:alginate (FIG. 3B) and TrzN:chitosan (FIG. 3C) are shown. These images were obtained using an electron detector that recorded back scattered electrons, so materials with a higher atomic number typically appear brighter and lighter in color. The TrzN:alginate appears lighter due to the Ca2+ ions in the alginate having a higher atomic number than the carbon, oxygen, and hydrogens that make up the composition of the chitosan. Quantitating the reaction of atrazine with each of the TrzN biomaterials is difficult as the exact quantity of encapsulated enzyme is not known; however, assuming that ˜99% of the enzyme used was encapsulated at least <1% of the solution state activity is retained at 25° C. Protein denaturation or inaccessibility to enzyme buried within the biomaterial likely results in reduced activity upon encapsulation of the enzyme. Even so, these biomolecular nanocomposites display the expected enzymatic properties including substrate recognition as TrzN in solution.

### Example 2—Proteolytic Digestion of Soluble TrzN and the TrzN:Alginate, TrzN:Chitosan, and TrzN:Sol-Gel Biomaterials

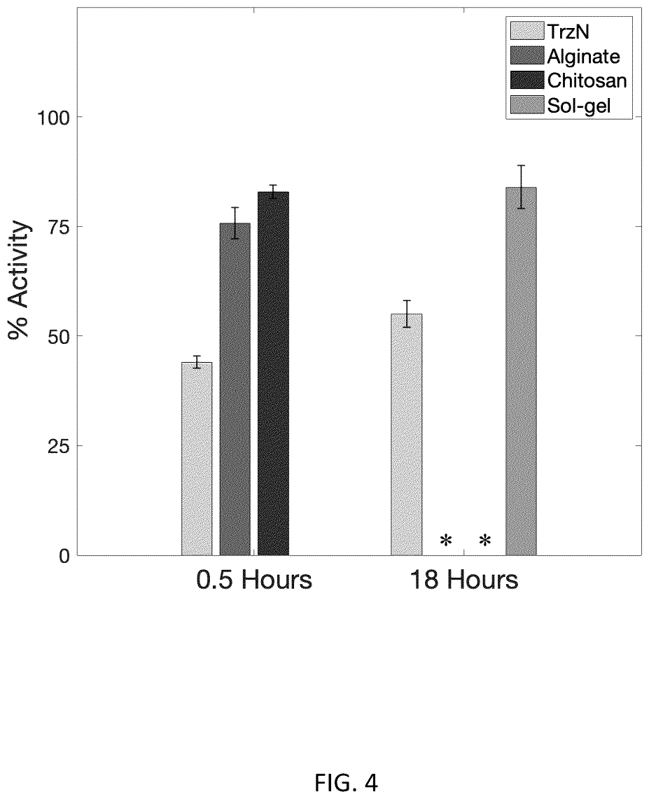

To ensure that the substrate has access to the fully immobilized enzyme and not enzyme that is simply adhered to the surface, each of the biomaterials were treated with trypsin to proteolytically digest all surface accessible protein (FIG. 4). TrzN:alginate and TrzN:chitosan were digested with trypsin for 30 min at 35° C. with constant shaking at 200 rpm. The TrzN:alginate and TrzN:chitosan biomaterials retained 75 and 80±5% of their original activity compared to a TrzN enzyme solution sample, which retained 55±5% of its activity. On the other hand, the TrzN:sol-gel biomaterial retained 85±5% of its activity even after 18-hrs in the presence of trypsin at 35° C. compared to a TrzN enzyme control sample, which only retained 45±5% of its activity over the same time period. These data indicate that substrate has access to the TrzN enzyme entrapped in the interior of each biomaterial, which is an active catalyst but protected from proteolytic digestion. Even though the TrzN:sol-gel and TrzN:chitosan provided comparable protection against proteolytic cleavage, the TrzN:sol-gel biomaterial was much more robust as it retained its activity after 18 hrs while TrzN:chitosan was only protective for ˜30 min. These data also indicate that surface bound enzyme is hydrolyzed, but interior TrzN is protected resulting in a catalyst that is more robust that the free enzyme.

### Example 3—Thermostability of Soluble TrzN and the TrzN:Alginate, TrzN:Chitosan, and TrzN:Sol-Gel Biomaterials

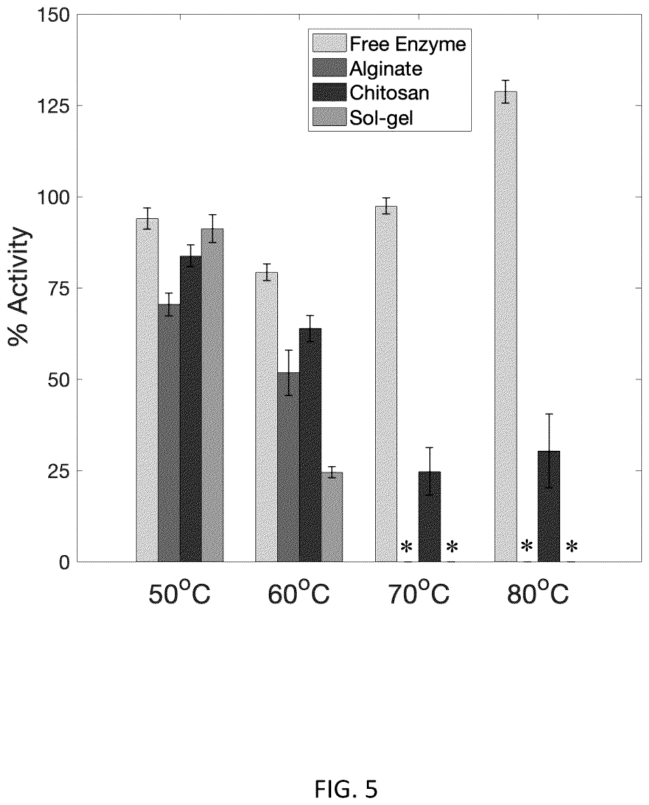

The thermostability of soluble TrzN enzyme and the TrzN:sol-gel, TrzN:alginate, and TrzN:chitosan biomaterials, was evaluated over a temperature range of 50-80° C. (FIG. 5). Both TrzN and each of the TrzN biomaterials is very thermally stable at 50° C. over 30 min incubation times with soluble TrzN exhibiting a kcat of 3.8±0.1 s−1, which is identical to that observed at 25° C. As the temperature is increased to 80° C., the soluble TrzN kcat increases to 5.0±0.2 s−1 while the TrzN:alginate and TrzN:sol-gel biomaterials were inactive after a 30 min incubation period. On the other hand, the TrzN:chitosan biomaterial retained ˜30% of its original activity at 80° ° C. over a 30 min incubation period. ICP analysis of the reaction buffers showed a significantly higher concentration of Ca2+ ions in the TrzN:alginate reaction buffer compared to those of TrzN:chitosan. These data suggest the polymer structure of the alginate tightened resulting in less substrate accessibility to the encapsulated enzyme while less Ca2+ ions in the chitosan reaction buffer indicates the chitosan was continuing to act as a barrier to protect the alginate matrix, resulting in increased activity in the chitosan coated biomaterial vs the alginate. Temperature has a direct relationship to the changing of the internal pore structure of alginate beads. Previous work reported a significant loss of moisture occurring during heat shock of alginate beads which created a dense porous structure. Therefore, the addition of the chitosan coating around the alginate matrix increased the thermostability of the alginate matrix.

### Example 4—Reusability and Long-Term Stability of the TrzN:Biomaterials

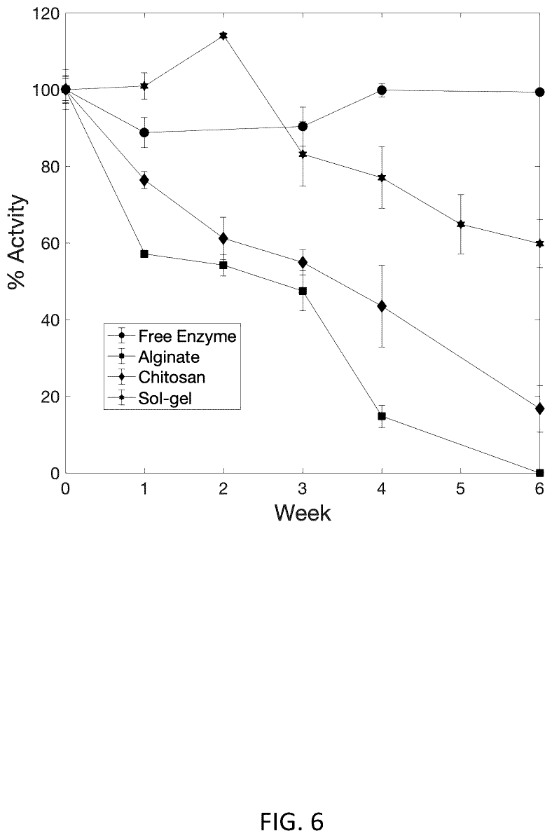

For commercial applications, a biocatalyst must be reusable and have long-term stability. With this is mind, we sought to investigate whether each of the biomaterials could be recycled in subsequent reactions (FIG. 6). After every use, each of the TrzN biomaterials were thoroughly washed with HEPES buffer, pH 7.0 to remove residual atrazine and hydroxyatrazine then stored in this buffer until they were submitted to the same reaction conditions. The conversion of atrazine to hydroxyatrazine decreases over a six-week period for each of the TrzN biomaterials with ˜65% of the initial activity remaining for TrzN:sol-gel but <20% for TrzN:chitosan and no observable activity for the TrzN:alginate biomaterial. Remarkably, soluble TrzN can also be stored at 4° C. in buffer without significant loss of activity over a six-week period (FIG. 6).

The loss in activity in the biomaterials compared to soluble TrzN may be due to enzyme loss during extended storage in an aqueous solution. For example, the TrzN:alginate biomaterial lost ˜70% of the original amount of TrzN encapsulated while the TrzN:chitosan biomaterial lost ˜55%. As previously noted, alginate beads tend to be “leaky” and while chitosan coating helps to decrease enzyme loss it does not stop it. The TrzN:sol-gel biomaterial also lost enzyme over the course of the six-week experiment but to a significantly smaller extent than either the TrzN:alginate or TrzN:chitosan biomaterials. These data indicate that enzyme remaining in the biomaterials stayed active and the loss in activity shown in these experiments was due to the loss of immobilized enzyme, rather than enzymatic denaturation within the matrix or product build up within the matrix hindering the diffusivity of the substrate to the enzyme. The cyclical reusability of TrzN:sol-gel over ten reactions revealed no loss in activity adding to the conclusion that the enzyme loss in extended storage was the cause of activity loss. Overall, the TrzN:sol-gel biomaterial performed the best and encapsulation of TrzN in a sol-gel provides a matrix for the storage of TrzN that can be stored for extended periods of time and can be recycled, stored, and reused.

### Example 5—Stability of the Soluble TrzN and TrzN:Biomaterials in Organic Co-Solvent

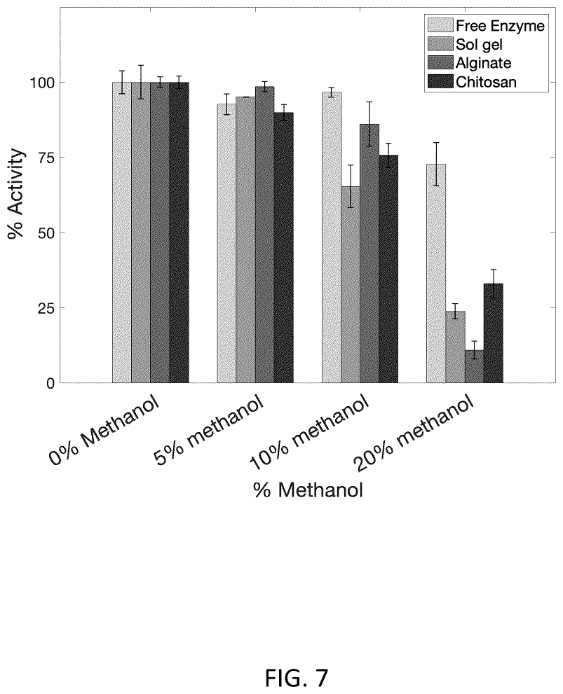

Atrazine presents itself traditionally in watersheds containing negligible amounts of organic solvents. However, at higher concentrations atrazine solubility becomes problematic and requires organic solvents. Thus, understanding the biomaterial's function in solutions with organic solvents is important in exploring their potential as bioremediation catalysts as enzymes typically denature when exposed to organic solvents. The activity of the TrzN:alginate, TrzN:chitosan, and TrzN:sol-gel biomaterials were examined in 5:95, 10:90, and 20:80 MeOH:water solvent mixtures (FIG. 7). At 5 and 10% methanol solution, each of the biomaterials retained at least 70% of the soluble TrzN enzyme but all exhibited a significant decrease in activity at 20% methanol with the TrzN:alginate biomaterial performing the worst with only 10% residual activity. At higher methanol concentrations, both the TrzN:alginate and TrzN:chitosan biomaterials lost all activity, however, the TrzN:sol-gel biomaterial retained ˜5% of its residual activity in a 70:30 MeOH:water solution. For comparison purposes, soluble TrzN exhibits ˜75% of its native enzyme's activity in 20:80 MeOH:water mixtures. The poorer performance in organic solvents for the TrzN:alginate and TrzN:chitosan biomaterials is due to increased Ca2+ ion concentration in the reaction solution, determined via ICP. Previously, it was reported that ethanol in concentrations >15% reduces the hydrodynamic volume of alginate beads resulting in a tightening of the polymer chains, which restricts substrate access to an encapsulated enzyme. Therefore, of the three biomaterials tested, sol-gel encapsulation clearly performs the best in stabilizing against increasing protic solvents.

### Example 6—Stability of the Soluble TrzN and TrzN:Biomaterials at Nonphysiological pH Values

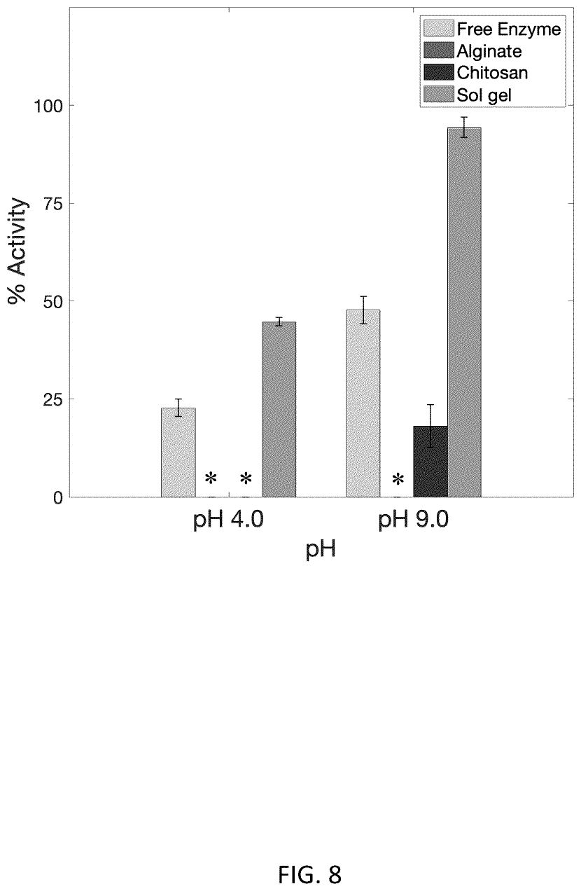

Typically, when using biomaterials in aqueous bioremediation strategies, the engineered material is expected to perform in the conditions presented within the watershed of interest. Although watersheds typically present a pH at ˜7, outside influences such as polluted runoff can cause the pH to fluctuate outside of the neutral range. With this in mind, it is important to investigate the activity of TrzN:alginate, TrzN:chitosan, and TrzN:sol-gel biomaterials in pH conditions at both high (pH 9.0) and low (pH 4.0) (FIG. 8). At pH 4.0, (50 mM citric acid buffer), both the TrzN:alginate and TrzN:chitosan biomaterials showed no activity while the TrzN:sol-gel biomaterial exhibited ˜45% of its residual activity. At pH 9.0, (50 mM glycine buffer) TrzN:alginate exhibited no detectable activity while the TrzN:chitosan and TrzN:sol-gel biomaterials retained ˜20 and 95% of their residual activities, respectively. For comparison purposes, soluble TrzN exhibits 25 and 45% of its residual activities at pH 4.0 and 9.0, respectively. Therefore, sol-gel encapsulation clearly affords significant stabilization against both increased and decreased proton ion concentrations, compared to the enzyme solutions, likely due to the strong electrostatic interaction between the silica sol and TrzN

### Example 7—Materials and Methods

Materials. Sodium alginate was purchased from SPECTRUM CHEMICAL MFG CORP. Chitosan, Atrazine, Tetramethyl orthosilicate (TMOS, ≥99%), and Type I Trypsin from bovine pancreas were both purchased from SIGMA-ALDRICH. All reagents were of the highest purity available and received without further purification.

Expression and purification of TrzN. The gene from Arthrobacter aurescens TC1 that encodes for TrzN with the D38N, L131P, and A159V mutations was synthesized with optimized E. coli codon usage that includes a polyhistidine (His6) affinity tag engineered onto the N-terminus between NdeI and XhoI restriction site of the kanamycin resistant pET28a(+) to create the TrzN/pET28a(+) plasmid. The TrzN/pET28a(+) plasmid was transformed into E. coli BL21(DE3) competent cells (STRATAGENE) for expression. A 100 mL LB-Miller starter culture was inoculated from a single colony with 50 μg/mL of kanamycin. A 9 L culture was inoculated from this starter culture using 10 mL/liter supplemented with 5 μM isopropyl ß-d-1-thiogalactopyranoside (IPTG) and grown at 37° C. for 48 h. Cells were harvested by centrifugation at 7000 rpm 4° C., for 15 min. The cells were resuspended at 2 mL per gram of buffer A (50 mM NaH2PO4, 500 mM NaCl, 10 mM imidazole, pH 7.5) then lysed by sonication on ice in three 10 min (30 s on, 45 s off) intervals using a 21W MISONEX sonicator 3000. Cell debris was removed by centrifugation at 17,500 rpm, 4° C., for 40 min. The protein was purified by immobilized metal affinity chromatography (IMAC) using Ni-NTA (nickel-nitrilotriacetic acid) Superflow Cartridges (QIAGEN). The column was equilibrated with buffer A and the crude protein extract was loaded onto the IMAC column. Unbound protein was eluted with 15 column volumes (CV) of buffer A at a flow rate of 2 mL/min. Elution of TrzN was initiated with 15 CV of 3% buffer B (buffer A with 500 mM imidazole). Elution finished with a linear gradient (3-100%) of buffer B over 20 CVs at a flow rate of 2 mL/min. Peak fractions were pooled and resuspended in 50 mM HEPES pH 7.5 and concentrated with an AMICON Ultra-15 centrifugal filter device with a molecular weight cutoff (MWCO) of 30,000 (MILLIPORE). SDS gel page reveals a single polypeptide band at ˜51 kDa, consistent with previous studies. The protein concentration was determined by UV-Vis absorbance at 280 nm (61,670 Molar Absorptivity) and with a Coomassie (Bradford) Protein Assay Kit (THERMO SCIENTIFIC). Expression of TrzN and purification using immobilized metal affinity chromatography (IMAC) resulted in ˜10 mg/L of soluble TrzN enzyme.

Kinetic Activity Assay. Hydrolysis of atrazine by TrzN was quantified spectrophotometrically by continuously monitoring the decrease in absorbance at 264 nm (ε264=3.5 mM⁻¹ cm⁻¹) that accompanies atrazine dechlorination. This region contains no detectable product absorption. The activity of purified TrzN was determined by measuring the hydrolysis of a 150 μM atrazine solution in 0.1 M sodium phosphate buffer, pH 7.0. Assays were performed in a 1 mL quartz cuvette in triplicate on an AGILENT 8453 UV-visible spectrophotometer. One unit of enzyme activity was defined as the amount of enzyme that catalyzed the hydrolysis of 1 μmol of atrazine per minute at 35° C. Plots of the initial rate of hydrolysis of various concentrations of atrazine were fit to the Michaelis-Menten equation, which provided a kcat of 3.97±0.14 s⁻¹ and a km value of 43.09±2.9 μM, similar to those previously reported.

Immobilization of TrzN in alginate beads without and with a chitosan coating, and sol-gels. Sodium alginate powder was added to 50 mM sodium phosphate buffer to 1% (w/v) and heated to 50° C. with vigorous shaking. TrzN (5 mg) was added to 4.5 mL of the cooled alginate solution with gentle stirring and transferred to a 5 ml syringe with a 16 G needle. The TrzN:alginate mixture was dripped into 50 mL of a 1 M CaCl2 solution with stirring (200 rpm) at 4° C. After ˜2 hrs, a 1 mL sample was removed and a Coomassie (Bradford) Protein Assay Kit (THERMO SCIENTIFIC) was used to determine the amount of TrzN encapsulated into alginate beads. Next, 150 mL of nanopure water was added and the beads were stirred for another 30 minutes to stop the gelation process. The resulting beads were then filtered and washed three times with 20 mL of nanopure water. The beads air dried for ˜30 minutes and then stored overnight at 4° C. in 5 mL of 50 mM HEPES buffer, pH 7.0.

TrzN:alginate beads were also coated in chitosan. Briefly, a chitosan solution was prepared by dissolving 0.8 g of chitosan (SIGMA-ALDRICH) in 90 mL of nanopure water after which 200 μl of glacial acetic acid (fisher) was added to facilitate the dissolution. The solution was filtered, and the volume increased to 100 mL with nanopure water providing a final concentration of 0.8% (w/v). The pH was adjusted to 5.6 and TrzN:alginate beads were placed in 50 mL of this chitosan solution and stirred at 200 rpm for ˜45 minutes. After the coating process, the TrzN:chitosan beads were washed three times with 20 ml of nanopure water and dried in air for 30 minutes. The beads were stored overnight at 4° C. in 5 mL of 50 mM HEPES buffer, pH 7.

The sol consisted of 1.57 mL of TMOS, 0.350 mL nanopure (18Ω) H₂O, and 0.011 mL of 0.040 M HCl. The mixture was sonicated on ice for 30 minute, and then left on ice for ˜1 hour prior to the addition of TrzN. An equal volume of TMOS sol (0.100 mL) was mixed with an equal volume of TrzN (2.5 mg) in 50 mM Tris-HCl, pH 7.5. The mixture was left on ice until gelation occurred creating TrzN:sol-gel monoliths. The monoliths were washed three times with 0.400 mL of 50 mM Tris-HCl, pH 7.5 (sol-gel buffer) and stored at 4° C. in 0.400 mL of the same buffer. The next day, the aged monoliths were crushed with a metal spatula to produce a heterogenous material and then washed three times with 0.400 mL of the sol-gel buffer. The wash and storage buffers were collected and tested for protein loss using the Coomassie (Bradford) Protein Assay kit.

Kinetic characterization of the TrzN:Alginate, TrzN:Chitosan, and TrzN:sol-gel biomaterials. The activity of the TrzN:alginate, TrzN:chitosan, and TrzN:sol-gel biomaterials was determined by measuring the hydrolysis of atrazine by continuously monitoring the decrease in absorbance at 264 nm (ε264=3.5 mM−1 cm−1) that accompanies atrazine dechlorination. A solution of 50 μM atrazine in 50 mM HEPES pH 7.0 at 25 or 35° C. was reacted with TrzN:alginate, TrzN:chitosan, and TrzN:sol-gel with constant stirring at 200 rpm. Aliquots of the reaction mixture (0.3 mL) were taken at fixed time intervals and the hydrolysis of atrazine analyzed. The specific activity (U/mg) of each biomaterial was calculated from the reaction rate (μmol/L/min), the amount of TrzN immobilized, and the volume of the reaction. The concentration of atrazine produced was determined using standard curves of absorbance versus known atrazine concentrations.

Proteolytic digestion of soluble TrzN, TrzN:alginate, TrzN:chitosan, and TrzN:sol-gel. Trypsin digestion of soluble TrzN (0.007 mg), TrzN:alginate beads (5 mg of TrzN), TrzN:chitosan beads (5 mg TrzN), and TrzN:sol-gel (2.5 mg TrzN) was performed at a ratio of 2:1 trypsin to TrzN in 50 mM Tris-HCl and 1 mM CaCl2 pH 7.6 (trypsin reaction buffer). TrzN:alginate and TrzN:chitosan was reacted for 30 min at 25° C. with constant stirring at 200 rpm while TrzN:sol-gel was reacted for 18 hours at 35° C. with constant stirring at 200 rpm. Along with the digested biomaterial samples, a control utilizing TrzN in trypsin reaction buffer was also incubated in an identical fashion. Following digestion, TrzN:alginate and TrzN:chitosan samples were filtered and washed three times with 20 mL of nanopure water while TrzN:sol-gel samples were filtered and washed three times with 0.4 mL sol-gel buffer to remove trypsin and digestion products. The biomaterials and digested TrzN were assayed for catalytic activity using standard assay conditions.

Recycling experiments for TrzN:alginate, TrzN:chitosan and TrzN:sol-gel. A 20 mL solution of 50 μM atrazine in 50 mM HEPES, pH 7.0 at 25° C. and reacted with TrzN:alginate or TrzN:chitosan for 6 hrs after which an aliquot (0.3 mL) was removed and the amount of atrazine dechlorination determined. The product mixture was then decanted, and the resulting biomaterial washed with 20 mL nanopure water. A 1 mL aliquot was taken of the reaction buffer to test for protein loss from the biomaterial using the Coomassie (Bradford) Protein Assay Kit (Thermo Scientific). The TrzN:alginate and TrzN:chitosan beads were stored at 4° C. in 5 mL of 50 mM HEPES pH 7.0 and reused on a weekly basis for six weeks.

A 5 mL solution of 50 μM atrazine in 50 mM HEPES pH 7.0 at 35° C. was reacted with TrzN:sol-gel for 1 hour after which the solution was centrifuged at 4000 rpm for 5 minutes. An aliquot (0.3 mL) of the supernatant was removed and the amount of atrazine dechlorination determined. A 1 mL aliquot of the supernatant was also taken to test for protein loss from the biomaterial using the Coomassie (Bradford) Protein Assay Kit (THERMO SCIENTIFIC). The TrzN:sol-gel biomaterial was washed three times with 0.4 mL of sol-gel buffer and stored at 4° C. in 0.4 mL of sol-gel buffer and reused on a weekly basis for six weeks. The reaction was also repeated on a different TrzN:sol-gel biomaterial 10 times over a 1 Hr time period as described above resulting in 10 separate cycles with the same sample of TrzN:sol-gel biomaterial.

Activity of soluble and immobilized TrzN in organic co-solvents. The activity of soluble TrzN with 5, 10, and 20% (v/v) methanol as the organic co-solvent toward atrazine was measured spectrophotometrically at 25° C. as described above. Each measurement was taken in triplicate. The degradation of atrazine using TrzN:Alginate, TrzN:Chitosan, and TrzN:sol-gel with organic co-solvents at 5, 10, and 20% (v/v) was carried out in their respective reaction conditions described previously. Aliquots, 1 mL, were taken at the start and end of the reaction and analyzed spectrophotometrically as described above.

Activity of soluble and immobilized TrzN at varying pH values. The activity of soluble and encapsulated TrzN in 50 mM citric acid, pH 4.0 and 50 mM glycine buffer, pH 9.0, towards atrazine was measured spectrophotometrically as described above. Each measurement was taken in triplicate. Samples, 1.5 mL, were taken at the end of the reactions and tested for protein loss using Coomassie (Bradford) Protein Assay Kit (THERMO SCIENTIFIC) and sent for ICP-MS analysis.

Thermostability of soluble and immobilized TrzN. The thermostability of TrzN, TrzN:alginate, and TrzN:chitosan was determined incubating each for 30 min at 50° C., 60° C., 70° C., and 80° C. TrzN:alginate and TrzN:chitosan materials were suspended in 5 mL of 50 mM HEPES pH 7.0 and after the incubation period, a 1.5 mL sample was collected for ICP-MS and protein analysis using the Coomassie (Bradford) Protein Assay Kit (THERMO SCIENTIFIC). Residual activity of the free enzyme and encapsulated TrzN was determined spectrophotometrically. The thermostability of the TrzN:sol-gel biomaterial was evaluated over a temperature range of 50° C.-80° C. with incubation times of 0, 30 min, 60 min, 180 min, 300 min, 420 min, and 600 minutes. After the incubation period was complete, the residual activity was determined spectrophotometrically.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

### REFERENCES

Pathak, R. K.; Dikshit, A. K. Atrazine and Human Health. Int. J. Ecosyst. 2012, 1 (1), 14-23. doi.org/10.5923/j.ije.20110101.03.
de Albuquerque, F. P.; de Oliveira, J. L.; Moschini-Carlos, V.; Fraceto, L. F. An Overview of the Potential Impacts of Atrazine in Aquatic Environments: Perspectives for Tailored Solutions Based on Nanotechnology. Sci. Total Environ. 2020, 700. doi.org/10.1016/j.scitotenv.2019.134868.
Solomon, K. R.; BAKER, D. B.; RICHARDS, R. P.; DIXON, K. R.; KLAINE, S. J.; POINT, T. W. LA; KENDALL, R. J.; WEISSKOPF, C. P.; GIDDINGS, J. M.; GIESY, J. P.; LENWOOD W. HALL, J.; WILLIAMS, W. M. Ecological Risk Assessment of Atrazine In. Environ. Toxicol. 1996, 15 (1), 31-76.
Beaulieu, M.; Cabana, H.; Taranu, Z.; Huot, Y. Predicting Atrazine Concentrations in Waterbodies across the Contiguous United States: The Importance of Land Use, Hydrology, and Water Physicochemistry. Limnol. Oceanogr. 2020, 2966-2983.doi.org/10.1002/Ino.11568.
Graymore, M.; Stagnitti, F.; Allinson, G. Impacts of Atrazine in Aquatic Ecosystems. Environ. Int. 2001, 26 (7-8), 483-495. doi.org/10.1016/S0160-4120(01)00031-9.
Mudhoo, A.; Garg, V. K. Sorption, Transport and Transformation of Atrazine in Soils, Minerals and Composts: A Review. Pedosphere 2011, 21 (1), 11-25. doi.org/10.1016/S1002-0160(10)60074-4.
Chernyak, S. M.; Rice, C. P.; McConnell, L. L. Evidence of Currently-Used Pesticides in Air, Ice, Fog, Seawater and Surface Microlayer in the Bering and Chukchi Seas. Mar. Pollut. Bull. 1996, 32 (5), 410-419. doi.org/10.1016/0025-326X(95)00216-A.
Ocking, D. A. J. H.; Abbitt, K. I. J. B. AMPHIBIAN CONTRIBUTIONS TO ECOSYSTEM SERVICES. Herpetol. Conserv. Biol. 2014, 9, 1-17.
Hayes, T. B.; Khoury, V.; Narayan, A.; Nazir, M.; Parka, A.; Brown, T.; Adame, L.; Chan, E.; Buchholz, D.; Stueve, T.; Gallipeau, S. Atrazine Induces Complete Feminization and Chemical Castration in Male African Clawed Frogs (Xenopus Laevis). Proc. Natl. Acad. Sci. U.S.A. 2010, 107 (10), 4612-4617. doi.org/10.1073/pnas.0909519107.
Liu, Z.; Wang, Y.; Zhu, Z.; Yang, E.; Feng, X.; Fu, Z.; Jin, Y. Atrazine and Its Main Metabolites Alter the Locomotor Activity of Larval Zebrafish (Danio Rerio). Chemosphere 2016, 148, 163-170. doi.org/10.1016/j.chemosphere.2016.01.007.
ATSDR. Public Health Statement Atrazine. Agency Toxic Subst. Regist. 2015, No. September, 5-8.
Rostami, S.; Jafari, S.; Moeini, Z.; Jaskulak, M.; Keshtgar, L.; Badeenezhad, A.; Azhdarpoor, A.; Rostami, M.; Zorena, K.; Dehghani, M. Current Methods and Technologies for Degradation of Atrazine in Contaminated Soil and Water: A Review. Environ. Technol. Innov. 2021, 24, 102019. doi.org/10.1016/j.eti.2021.102019.
Shapir, N.; Pedersen, C.; Gil, O.; Strong, L.; Seffernick, J.; Sadowsky, M. J.; Wackett, L. P. TrzN from Arthrobacter Aurescens TC1 Is a Zinc Amidohydrolase. J. Bacteriol. 2006, 188 (16), 5859-5864. doi.org/10.1128/JB.00517-06.
Mu, Y.; Zhan, G.; Huang, C.; Wang, X.; Ai, Z.; Zou, J.; Luo, S.; Zhang, L. Dechlorination-Hydroxylation of Atrazine to Hydroxyatrazine with Thiosulfate: A Detoxification Strategy in Seconds. Environ. Sci. Technol. 2019, 53 (6), 3208-3216. doi.org/10.1021/acs.est.8b06351.
Sheldon, R. A. Enzyme Immobilization: The Quest for Optimum Performance. Adv. Synth. Catal. 2007, 349 (8-9), 1289-1307. doi.org/10.1002/adsc.200700082.
Martinez, S.; Kuhn, M. L.; Russell, J. T.; Holz, R. C.; Elgren, T. E. Acrylamide Production Using Encapsulated Nitrile Hydratase from Pseudonocardia thermophila in a Sol-Gel Matrix. J. Mol. Catal. B Enzym. 2014, 100, 19-24. doi.org/10.1016/j.molcatb.2013.11.014.
Zhang, Y.; Dong, X.; Jiang, Z.; Cao, B.; Ge, S.; Hu, M. Assessment of the Ecological Security of Immobilized Enzyme Remediation Process with Biological Indicators of Soil Health. Environ. Sci. Pollut. Res. 2013, 20 (8), 5773-5780. doi.org/10.1007/s11356-012-1455-2.
LI, Q.; LI, Y.; ZHU, X.; CAI, B. Isolation and Characterization of Atrazine-Degrading Arthrobacter Sp. AD26 and Use of This Strain in Bioremediation of Contaminated Soil. J. Environ. Sci. 2008, 20 (10), 1226-1230. doi.org/10.1016/S1001-0742(08)62213-5.
Jackson, C. J.; Coppin, C. W.; Carr, P. D.; Aleksandrov, A.; Wilding, M.; Sugrue, E.; Ubels, J.; Paks, M.; Newman, J.; Peat, T. S.; Russell, R. J.; Field, M.; Weik, M.; Oakeshott, J. G.; Scott, C. 300-Fold Increase in Production of the Zn2+-Dependent Dechlorinase TrzN in Soluble Form via Apoenzyme Stabilization. Appl. Environ. Microbiol. 2014, 80 (13), 4003-4011. doi.org/10.1128/AEM.00916-14.
Kim, S.; Jeong, C.; Cho, S.; Kim, S. B. Effects of Thermal Treatment on the Physical Properties of Edible Calcium Alginate Gel Beads: Response Surface Methodological Approach. Foods 2019, 8 (11). doi.org/10.3390/foods8110578.
Fareez, I. M.; Lim, S. M.; Mishra, R. K.; Ramasamy, K. Chitosan Coated Alginate-Xanthan Gum Bead Enhanced PH and Thermotolerance of Lactobacillus Plantarum LAB12. Int. J. Biol. Macromol. 2015, 72, 1419-1428. doi.org/10.1016/j.ijbiomac.2014.10.054.
Won, K.; Kim, S.; Kim, K. J.; Park, H. W.; Moon, S. J. Optimization of Lipase Entrapment in Ca-Alginate Gel Beads. Process Biochem. 2005, 40 (6), 2149-2154. doi.org/10.1016/j.procbio.2004.08.014.
Bedade, D. K.; Sutar, Y. B.; Singhal, R. S. Chitosan Coated Calcium Alginate Beads for Covalent Immobilization of Acrylamidase: Process Parameters and Removal of Acrylamide from Coffee. Food Chem. 2019, 275 (June 2018), 95-104. doi.org/10.1016/j.foodchem.2018.09.090.
Im, J. K.; Cho, Y. C.; Noh, H. R.; Yu, S. J. Geographical Distribution and Risk Assessment of Volatile Organic Compounds in Tributaries of the Han River Watershed. Agronomy 2021, 11 (5). doi.org/10.3390/agronomyl1050956.
Hermansson, E.; Schuster, E.; Lindgren, L.; Altskar, A.; Strom, A. Impact of Solvent Quality on the Network Strength and Structure of Alginate Gels. Carbohydr. Polym. 2016, 144, 289-296. doi.org/10.1016/j.carbpol.2016.02.069.
Baker, J. P.; Bernard, D. P.; Christensen, S. W.; Sale, M. J.; Freda, J.; Heltcher, K.; Marmorek, D.; Rowe, L.; Scanlon, P.; Suter, G.; Warren-Hicks, W. J.; Welbourn, P. M. Biological Effects of Changes in Surface Water Acid-Base Chemistry. Acidic Depos. State Sci. Technol. 1990.
Li, F. Y.; Xing, Y. J.; Ding, X. Immobilization of Papain on Cotton Fabric by Sol-Gel Method. Enzyme Microb. Technol. 2007, 40 (7), 1692-1697. doi.org/10.1016/j.enzmictec.2006.09.007.
Náray-Szabó, G. Electrostatic Effects in Proteins. Period. Biol. 1999, 101 (4), 325-331. doi.org/10.1146/annurev.biophys.14.1.387.
Seffernick, J. L.; Reynolds, E.; Fedorov, A. A.; Fedorov, E.; Almo, S. C.; Sadowsky, M. J.; Wackett, L. P. X-Ray Structure and Mutational Analysis of the Atrazine Chlorohydrolase TrzN. J. Biol. Chem. 2010, 285 (40), 30606-30614. doi.org/10.1074/jbc.M110.138677.
DeGroot, A. R.; Neufeld, R. J. Encapsulation of Urease in Alginate Beads and Protection from α-Chymotrypsin with Chitosan Membranes. Enzyme Microb. Technol. 2001, 29 (6-7), 321-327. doi.org/10.1016/S0141-0229(01)00393-3.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. A method of protecting a bioremediation enzyme, comprising:
combining a support material with a plurality of enzyme molecules, wherein the enzyme molecules are selected from bacterial, archaea, and eukaryote enzyme molecules; and
allowing the combination to form a gel, wherein at least a portion of the enzyme molecules are encapsulated within the gel and not accessible to free protease.

2. The method of claim 1, wherein the enzyme is a dehalogenase.

3. The method of claim 1 or claim 2, wherein the support material is one or more of alginate, chitosan, and tetramethylorthosilicate (TMOS).

4. The method of any one of claims 1-3, wherein the gel is a sol-gel.

5. The method of any one of claims 1-4, wherein the gel is formed into beads and coated.

6. The method of any one of claims 1-5, wherein the encapsulated enzyme is more stable than the enzyme in solution.

7. The method of any one of claims 1-5, wherein the encapsulated enzyme is more resistant to a protic solvent than the enzyme free in solution.

8. The method of any one of claims 1-5, wherein the encapsulated enzyme is more resistant to an alcohol solvent than the enzyme in solution.

9. The method of any one of claims 1-5, wherein the encapsulated enzyme is more resistant to elevated temperature than the enzyme in solution.

10. The method of any one of claims 1-5, wherein the encapsulated enzyme is more resistant to a pH greater than 7.5 or less than 6.5 compared to the enzyme in solution.

11. The method of any one of claims 1-10, wherein the enzyme is selected from triazine hydrolase (TrzN) and chlorothalonil dehalogenase (Chd).

12. The method of any one of claims 1-11, wherein the enzyme is TrzN from Arthrobacter or Pseudomonas.

13. A composition for stabilizing an enzyme, comprising:
a support material comprising one or more of alginate, chitosan, and tetramethylorthosilicate (TMOS); and
a dehalogenase, wherein at least a portion of the dehalogenase is encapsulated within the support material.

14. The composition of claim 13, wherein the dehalogenase is bacterial, archaea, or eukaryotic.

15. The compositions of any one of claims 13-14, wherein the dehalogenase is selected from triazine hydrolase (TrzN) and chlorothalonil dehalogenase (Chd).

16. The composition of any one of claims 13-15, wherein the encapsulated dehalogenase is more stable than the enzyme in solution.

17. The composition of any one of claims 13-16, wherein the encapsulated dehalogenase is more resistant to a one or more of a protic solvent, an alcohol, elevated temperature, a pH greater than 7.5 or less than 6.5 compared to the dehalogenase in solution.

18. The composition of any one of claims 13-17, wherein the dehalogenase is selected from triazine hydrolase (TrzN) and chlorothalonil dehalogenase (Chd).

19. The composition of any one of claims 13-18, wherein the dehalogenase is TrzN from Arthrobacter or Pseudomonas.

20. A system for decontaminating a fluid, comprising:
a bioreactor, having a lumen;
an inlet in fluid communication with the lumen of the bioreactor;
an outlet in fluid communication with the lumen of the bioreactor; and
a biomaterial within the lumen, wherein the biomaterial comprises;
a material selected from one or more of alginate, chitosan, and tetramethylorthosilicate; and
a bacterial, archaea, or eukaryotic dehalogenase, wherein at least a portion of the dehalogenase is encapsulated in the material.

21. The system of claim 20, wherein the dehalogenase is selected from triazine hydrolase (TrzN) and chlorothalonil dehalogenase (Chd).

22. The system of any one of claims 20-21, wherein the encapsulated dehalogenase is more stable than the enzyme in solution.

23. The system of any one of claims 20-22, wherein the encapsulated dehalogenase is more resistant to a one or more of a protic solvent, an alcohol, elevated temperature, a pH greater than 7.5 or less than 6.5 compared to the dehalogenase in solution.

24. The system of any one of claims 20-23, wherein the dehalogenase is TrzN from Arthrobacter or Pseudomonas.

25. The system of any one of claims 20-24, wherein fluid entering the bioreactor lumen at the inlet comprises a concentration of contaminant that is higher than fluid leaving the bioreactor lumen at the outlet.
